(19) United States
(12) Patent Application Publication
(10) Pub. No.: US 2017/0071837 A1
(43) Pub. Date: Mar. 16, 2017

(54) CLEANING COMPOSITIONS COMPRISING BIOSURFACTANTS IN A FOAM DISPENSER

(71) Applicant: Henkel AG & Co. KGaA, Duesseldorf (DE)

(72) Inventors: Heike Schelges, Willich (DE); Maria Tretyakova, Dortmund (DE)

(73) Assignee: Henkel AG & Co. KGaA, Duesseldorf (DE)

(21) Appl. No.: 15/257,652

(22) Filed: Sep. 6, 2016

(30) Foreign Application Priority Data
Sep. 14, 2015 (DE) 10 2015 217 502.2

(51) Int. Cl. A61K 8/41; B05B 11/00; A61K 8/04; A61K 8/60; A61Q 19/10

(52) U.S. Cl. CPC A61K 8/41; A61K 8/602; A61Q 19/10; A61K 8/046; B05B 11/30; A61K 2800/596; A61K 2800/87

(57) ABSTRACT

A cleaning agent, in particular a cosmetic cleaning agent, includes a surfactant content that is largely or only composed of biosurfactants, in combination with a foam dispenser.

### FIELD OF THE INVENTION

The present invention generally relates to a cleaning agent, and more particularly relates to a cosmetic cleaning agent, the surfactant content of which is largely or only composed of biosurfactants, in combination with a foam dispenser.

### BACKGROUND OF THE INVENTION

Aqueous cosmetic cleaning agents typically contain surfactant mixtures, which may include anionic, amphoteric, non-ionic and/or cationic surfactants. Cleaning agents that include these surfactants have good cleaning power and in particular good foaming behavior. However, most of these surfactants are completely or partly obtained from petrochemicals. It is, however, becoming ever more important for cosmetic ingredients to be sustainable and this is increasingly demanded by consumers and manufacturers of cosmetic cleaning agents.

Biosurfactants are surface-active substances of microbial origin that can be produced using a substrate of plant oils or sugar. Some of these substrates can be agricultural waste such as rice husks or wastewater from the sugar industry, and in such cases no basic materials for food production are wasted. Biosurfactants thus satisfy the requirements of sustainability since they are produced from renewable resources. They are used in domestic cleaning agents, washing detergents and dishwasher detergents (e.g. U.S. Pat. No. 5,520,839, DE 19600743 A1), as well as in various cosmetic cleaning agents (e.g. WO 2014/095367 A1, WO 2013/098066 A2).

WO 2014/095367 A1 discloses the use of biosurfactants in combination with anionic surfactants such as sodium lauryl ether sulfate. However, in this case at least 10 wt. %, in the examples up to 50 wt. %, of the surfactant content is still composed of anionic surfactants. Hitherto, the relatively poor foaming ability of biosurfactants has prevented them from being used as the entire surfactant content or as most of the entire surfactant content of cosmetic cleaning agents.

It is therefore desirable to provide cosmetic cleaning agents, the surfactant content of which is largely or only composed of surfactants produced from renewable resources, in an acceptable form for the consumer in terms of the foaming and washing behavior.

Furthermore, other desirable features and characteristics of the present invention will become apparent from the subsequent detailed description of the invention and the appended claims, taken in conjunction with this background of the invention.

### BRIEF SUMMARY OF THE INVENTION

A cleaning agent system includes a cleaning agent, 95 wt. % to 100 wt. % of the surfactant content of which is composed of biosurfactant(s); and a foam dispenser for applying the cleaning agent.

A cleaning agent or cleaning agent concentrate, which consists of water and a biosurfactant, is used as a cosmetic cleaning agent, wherein a preservative, a perfume or fragrance, a caring substance, a thickener or a combination thereof can optionally also be included in the cleaning agent, as a cosmetic cleaning agent.

### DETAILED DESCRIPTION OF THE INVENTION

The following detailed description of the invention is merely exemplary in nature and is not intended to limit the invention or the application and uses of the invention. Furthermore, there is no intention to be bound by any theory presented in the preceding background of the invention or the following detailed description of the invention.

It has surprisingly been found that this can be achieved by using a cleaning agent of which 95 wt. % to 100 wt. % of the surfactant content is composed of biosurfactant(s), and by using said agent in combination with a foam dispenser for applying the cleaning agent. More particularly, a cosmetic cleaning agent can be used by which extremely satisfactory foaming behavior and washing behavior can be achieved for the consumer whilst being exceptionally pleasant to use, and sustainability and biodegradability requirements for the ingredients therein also being satisfied at the same time.

The Present Invention Relates to:

1. A cleaning agent system comprising:
a cleaning agent, 95 wt. % to 100 wt. % of the surfactant content of which is composed of biosurfactant(s), and
a foam dispenser for applying the cleaning agent.
   2. The cleaning agent system according to point 1, wherein the cleaning agent is provided as a concentrate separately from the foam dispenser.
   3. The cleaning agent system according to point 1 or 2, wherein 99 wt. % to 100 wt. %, preferably 99.5 wt. % to 99.9 wt. %, of the surfactant content of the cleaning agent is composed of biosurfactant(s).
   4. The cleaning agent system according to one of the preceding points, which contains a glycolipid, a lipopeptide or a combination thereof as the biosurfactant.
   5. The cleaning agent according to one of the preceding points, which contains a rhamnolipid, a sophorolipid, a mannosylerythritol lipid, a surfactin, a fatty acyl glutamate, a fatty acyl glycinate or a combination thereof as the biosurfactant.
   6. The cleaning agent according to point 5, which contains a sophorolipid as the biosurfactant, preferably a mixture of the acidic form and lactone form, wherein 20 to 60 wt. % is in the acidic form.
   7. The cleaning agent according to point 5, which contains a rhamnolipid as the biosurfactant, preferably a mixture of mono- and dirhamnolipid, which are each derived from 3-hydroxydodecanoic acid and/or 3-hydroxyundecanoic acid.
   8. The cleaning agent system according to one of the preceding points, wherein the cleaning agent consists of water and the biosurfactant, wherein a preservative, a perfume or fragrance, a caring substance, a thickener or a combination thereof can optionally be contained.
   9. The cleaning agent system according to one of the preceding points, wherein the cleaning agent is formulated as a cosmetic cleaning agent, in particular as a cleaning agent for the face, body or hands, or as a concentrate thereof.
   10. The cleaning agent system according to one of the preceding points, wherein the cleaning agent contains only water as the carrier.
   11. The cleaning agent system according to one of the preceding points, wherein the cleaning agent or cleaning agent concentrate is provided in a bag, in particular a bag made of biodegradable material, and can be transferred into the foam dispenser in order to be used.
   12. The cleaning agent system according to one of the preceding points, wherein the foam dispenser is a foam pump.
   13. The cleaning agent system according to point 11, wherein an F2 foam pump or a Cuisipro® foam pump is used as the foam pump.
   14. The use of a cleaning agent or cleaning agent concentrate, which consists of water and a biosurfactant, wherein a preservative, a perfume or fragrance, a caring substance, a thickener or a combination thereof can optionally also be contained in the cleaning agent, as a cosmetic cleaning agent.

As one constituent, the cleaning agent system according to the invention includes a cleaning agent, the surfactant content of which mainly consists of biosurfactants. More precisely, 95 to 100 wt. %, more preferably 97 to 100 wt. %, even more preferably 99 wt. % to 100 wt. %, also preferably 99.5 to 99.9 wt. %, of the surfactant content of the cleaning agent is composed of biosurfactant(s).

Biosurfactants are understood to be substances that are formed by microorganisms and are often expelled from the cell. Like classic surfactants, biosurfactants are surface-active substances that reduce the surface tension of liquids and thereby promote the mixing of aqueous (hydrophilic) and water-repellent (hydrophobic) phases. Biosurfactants can be produced under gentle production conditions that require little energy. They are generally highly biodegradable and are very environmentally friendly. Moreover, they are not toxic, nor are any toxic byproducts produced during the production thereof. Carbohydrates, in particular sugar, e.g. glucose, and/or lipophilic carbon sources such as fats, oils, partial glycerides, fatty acids, fatty alcohols, long-chain saturated or unsaturated hydrocarbons, are used as raw materials for the microbial production of the biosurfactants. According to the invention, the biosurfactants are preferably biosurfactants produced by fermentation.

Biosurfactants include glycolipids, lipopeptides, lipoproteins, fatty acids, phospholipids, neutral lipids and polymeric surfactants (e.g. emulsan), which can all also be used in the present invention.

Glycolipids that can be used in the present invention are compounds in which one or more monosaccharide units are glycosidically bonded to a lipid moiety. Examples of glycolipids as biosurfactants that can be used according to the invention are rhamnolipids, sophorolipids, mannosylerythritol lipids and trehalose lipids. Among these, rhamnolipids, sophorolipids, mannosylerythritol lipids and combinations thereof are preferred.

Rhamnolipids are obtained from bacteria of the genus Pseudomonas, in particular from Pseudomonas aeruginosa, preferably when grown on hydrophobic substrates such as n-alkanes or plant oils. Other glycolipids, for example glucose lipids, cellobiose lipids or trehalose lipids, are produced in turn by other microorganisms on different substrates. According to the invention, mannosylerythritol lipids are also preferred glycolipid biosurfactants; they are produced by Pseudozyma sp., Candida antarctica and Ustilago sp. bacteria.

According to the invention, rhamnolipids have the following general formula:

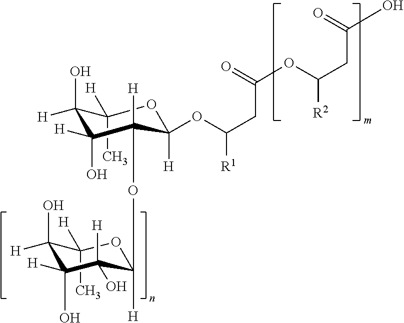

where m is 2, 1 or 0,
n is 1 or 0,
R¹ and R² are, independently of one another, the same or a different organic functional group having 2 to 24, preferably 5 to 13 carbon atoms, in particular a substituted or unsubstituted, branched or unbranched alkyl functional group, which can also be unsaturated, wherein the alkyl functional group is preferably a linear saturated alkyl functional group having 8 to 12 carbon atoms, more preferably is a nonyl or a decyl functional group or a mixture thereof.
Salts of these compounds are also included according to the invention.

In the present invention, the term “dirhamnolipid” is understood to mean compounds of the above formula or the salts thereof in which n is 1.

Accordingly, “monorhamnolipid” is understood in the present invention to mean compounds of the general formula or the salts thereof in which n is 0.

Mixtures of mono- and dirhamnolipids can preferably be used according to the invention. In this case, the ratio of monorhamnolipid to dirhamnolipid is preferably approximately 2:1 to 4:1, more preferably 2.5:1 to 3:1. Particularly preferred are those mixtures of mono- and dirhamnolipid in which, in the above formula, R¹ and R² independently represent a linear nonyl or decyl functional group. In the latter case, these are rhamnolipids that are each derived from 3-hydroxydodecanoic acid and/or 3-hydroxyundecanoic acid. Mixtures of this type can be obtained commercially under the name Rhamnolipid R90, R95 or R98 from Agae Technologies, USA, the number indicating the purity in each case. Rhamnolipid R90 can be used particularly preferably according to the invention.

If stronger foaming properties are desired, a cleaning agent comprising rhamnolipids as the single or a substantial constituent of the biosurfactants is preferred according to the invention. Therefore, preferred cleaning agents in which the content of the rhamnolipids is 50 to 100 wt. % of the biosurfactant content are included in embodiments of the present invention.

Sophorolipids are produced by fermentation using yeasts such as Candida bombicola (also known as Torulopsis bombicola), Yarrowia lipolytica, Candida apicola (Torulopsis apicola) and Candida bogoriensis, by growing said yeasts on sugars, hydrocarbons, plant oils or mixtures thereof.

Sophorolipids have the following formulae (1) (lactone form) and (2) (free acid), the two forms typically being provided in a mixture.

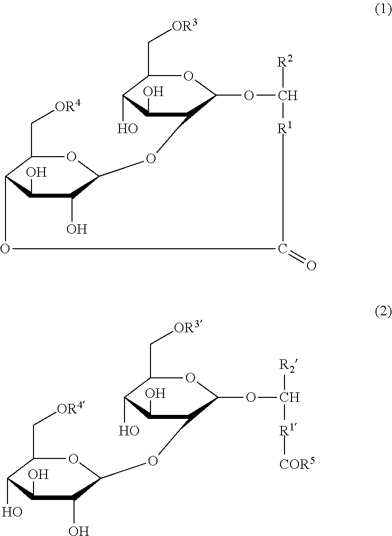

where R¹ and R^{1′} independently represent saturated hydrocarbon chains or single or multiple, in particular single, unsaturated hydrocarbon chains having 8 to 20, in particular 12 to 18 carbon atoms, more preferably 14 to 18 carbon atoms, which can be linear or branched and can comprise one or more hydroxy groups,
R² and R^{2′} independently represent a hydrogen atom or a saturated alkyl functional group or a single or multiple, in particular single, unsaturated alkyl functional group having 1 to 9 carbon atoms, more preferably 1 to 4 carbon atoms, which can be linear or branched and can comprise one or more hydroxy groups, and
R³, R^{3′}, R⁴ and R^{4′} independently represent a hydrogen atom or an acetyl group.

Sophorolipids in which R¹ and R^{1′} are single, unsaturated, linear hydrocarbon chains having 15 carbon atoms are preferred. It is also preferred for R² and R^{2′} to represent a methyl group or a hydrogen atom, even more preferably for each to represent a methyl group.

According to the invention, sophorolipids in which the acidic form and the lactone form are in a mixture are preferred, preferably approximately 20 to approximately 60 wt. % of the sophorolipids being in the acidic form and the remainder of the sophorolipids being in the lactone form.

In particular, sophorolipids are preferred in which compounds of the above formulae (1) and (2) are present in a mixture, where R¹ and R^{1′} are a single, unsaturated, linear hydrocarbon chain having 14 to 18 carbon atoms, even more preferably 15 carbon atoms, R³ and R⁴ represent an acetyl group, R^{3′} and R^{4′} represent a hydrogen atom and R² and R^{2′} represent a methyl group, and approximately 20 to 60 wt. % of the sophorolipids being in the acidic form.

Sophorolipids of this type can be obtained commercially, for example under the name Sopholiance S from Soliance. More precisely, the sophorolipid that can be obtained under the trade name Sopholiance S from Soliance is an approximately 60 wt. % sophorolipid solution and is, for example, obtained by fermenting Candida bombicola on rapeseed oil methyl ester and glucose (INCI: Candida bombicola/glucose/methyl rapeseed ferment (and) water). Sopholiance S is a preferred sophorolipid according to the invention. In Sopholiance, approximately 20 wt. % is present in the free acid form, in a mixture with the lactone form.

Mannosylerythritol lipids are glycolipids of the following general formula:

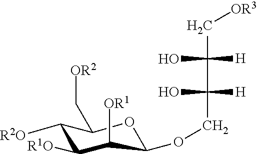

where R¹ independently represents fatty acid acyl groups having 4 to 24 carbon atoms, preferably 8 to 12 carbon atoms, R² independently represents a hydrogen atom or an acetyl group, and R³ represents a hydrogen atom or a fatty acid acyl group having 2 to 24 carbon atoms. A mannosylerythritol lipid that is suitable according to the invention can be obtained commercially under the name Ceramela-B (Toyobo) (INCI: Pseudozyma tsukubaensis/olive oil/glycerin/soy protein ferment).

The lipids and lipid derivatives substance group, to which in particular lipopeptides belong, is also included in the biosurfactants. In general, lipopeptides are synthesized non-ribosomally by the respective microorganisms, for example by Gram-positive bacteria, in particular of the genera Bacillus and Streptomyces, by Gram-negative bacteria, in particular of the genus Pseudomonas and Myxobacteria, and by filamentous plants. Normally, the peptide chains consist of two to forty amino acids, and can be linear, cyclic or branched. Unlike ribosomally synthesized peptide chains, lipopeptides often not only comprise proteinogenic L-amino acids as the monomer structural element, but also D-amino acids and carboxylic acids and/or all types of alpha-hydroxy carboxylic acids. The amino acids are mostly L-α- or D-α-amino acids, although β-, γ- or δ-amino acids can also be present, which can likewise also be in a D- or L-configuration. The peptide chains can also comprise other chemical modifications; in particular they can be glycolyzed, hydrolyzed, N-methylated or N-formylated. Common structural elements are also thiazoline rings and/or oxazoline rings in various oxidation stages. A known lipopeptide biosurfactant is surfactin, which has the following structure and is generally used as an alkali salt or ammonium salt:

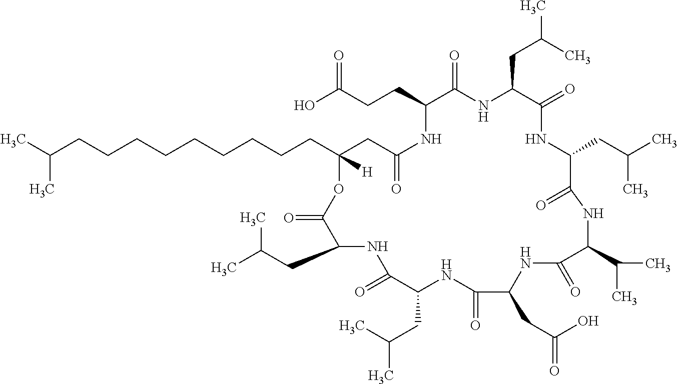 [SMILES: CC(C)CCCCCCCCC[C@@H]1CC(=O)N[C@@H](CCC(=O)O)C(=O)N[C@@H](CC(C)C)C(=O)N[C@H](CC(C)C)C(=O)N[C@@H](C(C)C)C(=O)N[C@@H](CC(=O)O)C(=O)N[C@H](CC(C)C)C(=O)N[C@@H](CC(C)C)C(=O)O1]

A surfactin that is suitable according to the invention can be obtained commercially from Kaneka.

The lipopeptides that can be preferably used as biosurfactants according to the invention also include fatty acyl glutamates. Said lipopeptides comprise the following general formula:

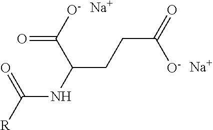

where R is a straight or branched alkyl chain having 5 to 21 carbon atoms, preferably 7 to 17 carbon atoms, more preferably 12 to 16 or 13 to 15 carbon atoms. Fatty acyl glutamates in the form of biosurfactants are generally provided in a mixture in which R has different chain lengths. The functional group R can also be hydroxylated, preferably by a single hydroxylation, in which case hydroxylation at β-position is preferred. Fatty acyl glutamates in the form of biosurfactants can, for example, be obtained from Modular Genetics, Inc., USA.

The lipopeptides that can preferably be used according to the invention as biosurfactants also include fatty acyl glycinates. These have the following general formula:

RC(O)NHCH₂CO₂X,

where
R is a straight or branched alkyl chain having 5 to 21 carbon atoms, preferably 7 to 17 carbon atoms, more preferably 12 to 16 or 13 to 15 carbon atoms, and
X is a cation, preferably an alkali metal cation or an ammonium cation, more preferably a sodium or ammonium cation, or —H.
   Fatty acyl glycinates in the form of biosurfactants can also be present in a mixture in which R can comprise different chain lengths.
   Fatty acyl glycinates in the form of biosurfactants can be obtained, for example, from Modular Genetics, Inc., USA.

According to the invention, cleaning agents that contain the following biosurfactants or biosurfactant combinations are preferred: rhamnolipid(s), rhamnolipid(s)+sophorolipid(s), rhamnolipid(s)+surfactin, rhamnolipid(s)+fatty acyl glutamate, rhamnolipid(s)+fatty acyl glycinate, rhamnolipid(s)+surfactin+sophorolipid(s), surfactin+sophorolipid(s), surfactin+fatty acyl glutamate, surfactin+fatty acyl glycinate. It is particularly preferable in this case for the rhamnolipid content of the biosurfactants to be 10 to 90 wt. %, more preferably 20 to 80 wt. %, more preferably 30 to 70 wt. % or 40 to 60 wt. %, in particular approximately 50 wt. %. In other embodiments, it is preferred for the surfactin content of the biosurfactants to be 10 to 90 wt. %, more preferably 20 to 80 wt. %, more preferably 30 to 70 wt. % or 40 to 60 wt. %, in particular approximately 50 wt. %.

The cleaning agent of the cleaning agent system according to the invention is in particular a cosmetic cleaning agent.

The cleaning agent, more particularly the cosmetic cleaning agent, contains water as the carrier. Other common carriers such as ethanol and glycol can be included in embodiments of the invention, but in terms of biodegradability and natural availability of the raw materials, it is preferred for the cleaning agent to only contain water as the carrier.

According to the invention, the cosmetic cleaning agent can contain additional conventional ingredients of cosmetic cleaning agents. Examples of common ingredients of this kind are fragrances, preservatives, caring substances and/or thickeners. However, the present invention is not restricted to these additional ingredients.

However, according to the invention, the cosmetic cleaning agent can also be formulated such that it contains no other constituents besides the biosurfactant or a biosurfactant combination and water, and optionally a preservative and/or fragrance in typical amounts. In particular, owing to the combination with a foam dispenser, it is possible according to the invention for the cleaning agent to only consist of biosurfactants and water.

Preferably, organic acids or salts thereof, such as sodium benzoate, can be used as preservatives. If a preservative is contained, it is preferably contained in an amount of 0 to 2 wt. %, preferably 0.05 to 1.5 wt. %, more preferably 0.1 to 1 wt. %, and most preferably 0.2 to 0.6 wt. %, in each case based on the total weight of the cleaning agent.

According to the invention, natural fragrances are preferred in particular as fragrances that can be contained in the cosmetic cleaning agent. If a fragrance is contained, it is preferably contained in an amount of 0 to 2 wt. %, preferably 0.05 to 1.5 wt. %, more preferably 0.1 to 1 wt. %, and most preferably 0.2 to 0.6 wt. %, in each case based on the total weight of the cleaning agent.

As a caring substance, the agent can, for example, contain oil components, preferably natural oil components such as plant oils and plant extracts, but also monosaccharides or oligosaccharides and/or lipids. Aloe vera extracts or olive oil are mentioned as examples. If a caring substance is contained, it is preferably contained in an amount of 0.1 to 10 wt. %, preferably 0.5 to 7 wt. %, more preferably 1 to 5 wt. %, in each case based on the total weight of the cleaning agent.

As thickeners, according to the invention, thickeners of plant origin are preferably contained, such as polysaccharides like celluloses (cellulose itself and derivatives thereof), alginic acids (and the corresponding physiologically acceptable salts thereof, the alginates), agar agar (with the polysaccharide agarose present as the main constituent in agar agar), starch fractions and derivatives such as amylose, amylopectin and dextrin, karaya gum, gellan gum, carob flour, gum arabic, dextrane, guar gum and xanthan gum or combinations thereof. Synthetic thickeners, such as polyacrylates, are preferably not contained.

Suitable cellulose derivatives are methyl celluloses, ethyl celluloses, hydroxyalkyl celluloses (such as hydroxyethyl cellulose), methyl hydroxyalkyl celluloses and carboxy methyl celluloses (INCI: cellulose gum) and the physiologically acceptable salts thereof.

If a thickener is contained, it is preferably contained in an amount of 0.01 to 30 wt. %, preferably 0.05 to 20 wt. %, more preferably 0.1 to 10 wt. %, in each case based on the total weight of the cleaning agent.

The above measurements for the preservatives, fragrances, caring substances and thickeners are each based on the concentrations when applying the cleaning agent. In the case of concentrates, the amounts may be accordingly higher.

The cleaning agent, more particularly a cosmetic cleaning agent, contains the biosurfactants in an amount of approximately 1 to 50 wt. %, preferably approximately 1 to 30 wt. %, more preferably approximately 2 to 20 wt. %, more preferably 2 to 15 wt. %, based on the total weight of the cleaning agent, the concentrations here being those when applying the cleaning agent.

The present invention relates to a cleaning agent system. In the process, the cleaning agent is provided in combination with a foam dispenser. According to the invention, the cleaning agent can be present in the foam dispenser in the form suitable for application. It is also possible according to the invention for the cleaning agent system to be designed such that the cleaning agent is initially marketed separately from the foam dispenser, for example in a plastics bag, and is only transferred into the foam dispenser by the user. In this case, the plastics bag is preferably made of a biodegradable material, for example a starch-based material. In particular if the cleaning agent is initially separate from the foam dispenser, it is preferable for the cleaning agent, which is initially provided separately, to be designed as a concentrate. The concentrate can be transferred into the foam dispenser at the user's premises and diluted with water. This embodiment in particular reduces transportation costs.

A concentrate diluted with water in the ratio of cleaning agent to water of approximately 1:2 to 1:4, preferably approximately 1:3 is preferred. Normal tap water or distilled water can be used for dilution.

According to the invention, a foam dispenser is understood to be a device having a container and a closure and by which liquid cosmetic cleaning agents, which are generally not foamed in the container, can be applied such that they foam upon exiting the closure. In particular, foam dispensers in which no propellant is needed to create the foam are included according to the invention. Accordingly, the cleaning agents in the present invention or the cleaning agent system preferably do(es) not contain any propellant. The design of the closure is thus substantially responsible for the creation of foam. Foam dispensers of this type are known. According to the invention, a foam pump is preferably used as a foam dispenser. Foam pumps can be obtained commercially. It has been shown that, according to the invention, the use of an F2 foam pump or a Cuisipro® foam pump delivers particularly good foaming results.

According to the invention, the cosmetic cleaning agent can be provided as a shampoo, shower gel, liquid hand soap, face cleaning agent, facial gel or other known cosmetic forms, or as a concentrate of each of these.

Overview in Tables:

Preferred cosmetic cleaning agents for the cleaning agent system according to the invention are set out below. All the information is given in wt. % and relates to the active ingredient concentration.

| | Formula 1 | Formula 2 | Formula 3 | Formula 4 |
|---|---|---|---|---|
| Rhamnolipid | 1 to 50 | 1 to 30 | 2 to 20 | 2 to 15 |
| Misc | total to 100 | total to 100 | total to 100 | total to 100 |
| | Formula 1a | Formula 2a | Formula 3a | Formula 4a |
| Rhamnolipid | 1 to 50 | 1 to 30 | 2 to 20 | 2 to 15 |
| Caring substance | 0.1 to 10 | 0.1 to 7 | 0.5 to 5 | 1 to 5 |
| Misc | total to 100 | total to 100 | total to 100 | total to 100 |
| | Formula 1b | Formula 2b | Formula 3b | Formula 4c |
| Rhamnolipid | 1 to 50 | 1 to 30 | 2 to 20 | 2 to 15 |
| Thickener | 0.01 to 30 | 0.05 to 20 | 0.1 to 15 | 0.1 to 10 |
| Misc | total to 100 | total to 100 | total to 100 | total to 100 |
| | Formula 1c | Formula 2c | Formula 3c | Formula 4c |
| Rhamnolipid | 1 to 50 | 1 to 30 | 2 to 20 | 2 to 15 |
| Caring substance | 0.1 to 10 | 0.1 to 7 | 0.5 to 5 | 1 to 5 |
| Thickener | 0.01 to 30 | 0.05 to 20 | 0.1 to 15 | 0.1 to 10 |
| Misc | total to 100 | total to 100 | total to 100 | total to 100 |
| | Formula 5 | Formula 6 | Formula 7 | Formula 8 |
| Rhamnolipid | 0.5 to 25 | 0.5 to 15 | 1 to 10 | 1 to 7.5 |
| Surfactin | 0.5 to 25 | 0.5 to 15 | 1 to 10 | 1 to 7.5 |
| Misc | total to 100 | total to 100 | total to 100 | total to 100 |
| | Formula 5a | Formula 6a | Formula 7a | Formula 8a |
| Rhamnolipid | 0.5 to 25 | 0.5 to 15 | 1 to 10 | 1 to 7.5 |
| Surfactin | 0.5 to 25 | 0.5 to 15 | 1 to 10 | 1 to 7.5 |
| Caring substance | 0.1 to 10 | 0.1 to 7 | 0.5 to 5 | 1 to 5 |
| Misc | total to 100 | total to 100 | total to 100 | total to 100 |
| | Formula 5b | Formula 6b | Formula 7b | Formula 8b |
| Rhamnolipid | 0.5 to 25 | 0.5 to 15 | 1 to 10 | 1 to 7.5 |
| Surfactin | 0.5 to 25 | 0.5 to 15 | 1 to 10 | 1 to 7.5 |
| Thickener | 0.01 to 30 | 0.05 to 20 | 0.1 to 15 | 0.1 to 10 |
| Misc | total to 100 | total to 100 | total to 100 | total to 100 |
| | Formula 5c | Formula 6c | Formula 7c | Formula 8c |
| Rhamnolipid | 0.5 to 25 | 0.5 to 15 | 1 to 10 | 1 to 7.5 |
| Surfactin | 0.5 to 25 | 0.5 to 15 | 1 to 10 | 1 to 7.5 |
| Caring substance | 0.1 to 10 | 0.1 to 7 | 0.5 to 5 | 1 to 5 |
| Thickener | 0.01 to 30 | 0.05 to 20 | 0.1 to 15 | 0.1 to 10 |
| Misc | total to 100 | total to 100 | total to 100 | total to 100 |
| | Formula 9 | Formula 10 | Formula 11 | Formula 12 |
| Rhamnolipid | 0.5 to 25 | 0.5 to 15 | 1 to 10 | 1 to 7.5 |
| Fatty acyl glutamate | 0.5 to 25 | 0.5 to 15 | 1 to 10 | 1 to 7.5 |
| Misc | total to 100 | total to 100 | total to 100 | total to 100 |
| | Formula 9a | Formula 10a | Formula 11a | Formula 12a |
| Rhamnolipid | 0.5 to 25 | 0.5 to 15 | 1 to 10 | 1 to 7.5 |
| Fatty acyl glutamate | 0.5 to 25 | 0.5 to 15 | 1 to 10 | 1 to 7.5 |
| Caring substance | 0.1 to 10 | 0.1 to 7 | 0.5 to 5 | 1 to 5 |
| Misc | total to 100 | total to 100 | total to 100 | total to 100 |
| | Formula 9b | Formula 10b | Formula 11b | Formula 12b |
| Rhamnolipid | 0.5 to 25 | 0.5 to 15 | 1 to 10 | 1 to 7.5 |
| Fatty acyl glutamate | 0.5 to 25 | 0.5 to 15 | 1 to 10 | 1 to 7.5 |
| Thickener | 0.01 to 30 | 0.05 to 20 | 0.1 to 15 | 0.1 to 10 |
| Misc | total to 100 | total to 100 | total to 100 | total to 100 |
| | Formula 9c | Formula 10c | Formula 11c | Formula 12c |
| Rhamnolipid | 0.5 to 25 | 0.5 to 15 | 1 to 10 | 1 to 7.5 |
| Fatty acyl glutamate | 0.5 to 25 | 0.5 to 15 | 1 to 10 | 1 to 7.5 |
| Caring substance | 0.1 to 10 | 0.1 to 7 | 0.5 to 5 | 1 to 5 |
| Thickener | 0.01 to 30 | 0.05 to 20 | 0.1 to 15 | 0.1 to 10 |
| Misc | total to 100 | total to 100 | total to 100 | total to 100 |
| | Formula 13 | Formula 14 | Formula 15 | Formula 16 |
| Rhamnolipid | 0.5 to 25 | 0.5 to 15 | 1 to 10 | 1 to 7.5 |
| Fatty acyl glycinate | 0.5 to 25 | 0.5 to 15 | 1 to 10 | 1 to 7.5 |
| Misc | total to 100 | total to 100 | total to 100 | total to 100 |
| | Formula 13a | Formula 14a | Formula 15a | Formula 16a |
| Rhamnolipid | 0.5 to 25 | 0.5 to 15 | 1 to 10 | 1 to 7.5 |
| Fatty acyl glycinate | 0.5 to 25 | 0.5 to 15 | 1 to 10 | 1 to 7.5 |
| Caring substance | 0.1 to 10 | 0.1 to 7 | 0.5 to 5 | 1 to 5 |
| Misc | total to 100 | total to 100 | total to 100 | total to 100 |
| | Formula 13b | Formula 14b | Formula 15b | Formula 16b |
| Rhamnolipid | 0.5 to 25 | 0.5 to 15 | 1 to 10 | 1 to 7.5 |
| Fatty acyl glycinate | 0.5 to 25 | 0.5 to 15 | 1 to 10 | 1 to 7.5 |
| Thickener | 0.01 to 30 | 0.05 to 20 | 0.1 to 15 | 0.1 to 10 |
| Misc | total to 100 | total to 100 | total to 100 | total to 100 |
| | Formula 13c | Formula 14c | Formula 15c | Formula 16c |
| Rhamnolipid | 0.5 to 25 | 0.5 to 15 | 1 to 10 | 1 to 7.5 |
| Fatty acyl glycinate | 0.5 to 25 | 0.5 to 15 | 1 to 10 | 1 to 7.5 |
| Caring substance | 0.1 to 10 | 0.1 to 7 | 0.5 to 5 | 1 to 5 |
| Thickener | 0.01 to 30 | 0.05 to 20 | 0.1 to 15 | 0.1 to 10 |
| Misc | total to 100 | total to 100 | total to 100 | total to 100 |
| | Formula 17 | Formula 18 | Formula 19 | Formula 20 |
| Rhamnolipid | 0.5 to 25 | 0.5 to 15 | 1 to 10 | 1 to 7.5 |
| Sophorolipid | 0.5 to 25 | 0.5 to 15 | 1 to 10 | 1 to 7.5 |
| Misc | total to 100 | total to 100 | total to 100 | total to 100 |
| | Formula 17a | Formula 18a | Formula 19a | Formula 20a |
| Rhamnolipid | 0.5 to 25 | 0.5 to 15 | 1 to 10 | 1 to 7.5 |
| Sophorolipid | 0.5 to 25 | 0.5 to 15 | 1 to 10 | 1 to 7.5 |
| Caring substance | 0.1 to 10 | 0.1 to 7 | 0.5 to 5 | 1 to 5 |
| Misc | total to 100 | total to 100 | total to 100 | total to 100 |
| | Formula 17b | Formula 18b | Formula 19b | Formula 20b |
| Rhamnolipid | 0.5 to 25 | 0.5 to 15 | 1 to 10 | 1 to 7.5 |
| Sophorolipid | 0.5 to 25 | 0.5 to 15 | 1 to 10 | 1 to 7.5 |
| Thickener | 0.01 to 30 | 0.05 to 20 | 0.1 to 15 | 0.1 to 10 |
| Misc | total to 100 | total to 100 | total to 100 | total to 100 |
| | Formula 17c | Formula 18c | Formula 19c | Formula 20c |
| Rhamnolipid | 0.5 to 25 | 0.5 to 15 | 1 to 10 | 1 to 7.5 |
| Sophorolipid | 0.5 to 25 | 0.5 to 15 | 1 to 10 | 1 to 7.5 |
| Caring substance | 0.1 to 10 | 0.1 to 7 | 0.5 to 5 | 1 to 5 |
| Thickener | 0.01 to 30 | 0.05 to 20 | 0.1 to 15 | 0.1 to 10 |
| Misc | total to 100 | total to 100 | total to 100 | total to 100 |
| | Formula 21 | Formula 22 | Formula 23 | Formula 24 |
| Rhamnolipid | 0.5 to 20 | 0.5 to 10 | 1 to 7.5 | 1 to 5 |
| Sophorolipid | 0.5 to 20 | 0.5 to 10 | 1 to 7.5 | 1 to 5 |
| Surfactin | 0.5 to 20 | 0.5 to 10 | 1 to 7.5 | 1 to 5 |
| Misc | total to 100 | total to 100 | total to 100 | total to 100 |
| | Formula 21a | Formula 22a | Formula 23a | Formula 24a |
| Rhamnolipid | 0.5 to 20 | 0.5 to 10 | 1 to 7.5 | 1 to 5 |
| Sophorolipid | 0.5 to 20 | 0.5 to 10 | 1 to 7.5 | 1 to 5 |
| Surfactin | 0.5 to 20 | 0.5 to 10 | 1 to 7.5 | 1 to 5 |
| Caring substance | 0.1 to 10 | 0.1 to 7 | 0.5 to 5 | 1 to 5 |
| Misc | total to 100 | total to 100 | total to 100 | total to 100 |
| | Formula 21b | Formula 22b | Formula 23b | Formula 24b |
| Rhamnolipid | 0.5 to 20 | 0.5 to 10 | 1 to 7.5 | 1 to 5 |
| Sophorolipid | 0.5 to 20 | 0.5 to 10 | 1 to 7.5 | 1 to 5 |
| Surfactin | 0.5 to 20 | 0.5 to 10 | 1 to 7.5 | 1 to 5 |
| Thickener | 0.01 to 30 | 0.05 to 20 | 0.1 to 15 | 0.1 to 10 |
| Misc | total to 100 | total to 100 | total to 100 | total to 100 |
| | Formula 21c | Formula 22c | Formula 23c | Formula 24c |
| Rhamnolipid | 0.5 to 20 | 0.5 to 10 | 1 to 7.5 | 1 to 5 |
| Sophorolipid | 0.5 to 20 | 0.5 to 10 | 1 to 7.5 | 1 to 5 |
| Surfactin | 0.5 to 20 | 0.5 to 10 | 1 to 7.5 | 1 to 5 |
| Caring substance | 0.1 to 10 | 0.1 to 7 | 0.5 to 5 | 1 to 5 |
| Thickener | 0.01 to 30 | 0.05 to 20 | 0.1 to 15 | 0.1 to 10 |
| Misc | total to 100 | total to 100 | total to 100 | total to 100 |
| | Formula 25 | Formula 26 | Formula 27 | Formula 28 |
| Surfactin | 0.5 to 25 | 0.5 to 15 | 1 to 10 | 1 to 7.5 |
| Sophorolipid | 0.5 to 25 | 0.5 to 15 | 1 to 10 | 1 to 7.5 |
| Misc | total to 100 | total to 100 | total to 100 | total to 100 |
| | Formula 25a | Formula 26a | Formula 27a | Formula 28a |
| Surfactin | 0.5 to 25 | 0.5 to 15 | 1 to 10 | 1 to 7.5 |
| Sophorolipid | 0.5 to 25 | 0.5 to 15 | 1 to 10 | 1 to 7.5 |
| Caring substance | 0.1 to 10 | 0.1 to 7 | 0.5 to 5 | 1 to 5 |
| Misc | total to 100 | total to 100 | total to 100 | total to 100 |
| | Formula 25b | Formula 26b | Formula 27b | Formula 28b |
| Surfactin | 0.5 to 25 | 0.5 to 15 | 1 to 10 | 1 to 7.5 |
| Sophorolipid | 0.5 to 25 | 0.5 to 15 | 1 to 10 | 1 to 7.5 |
| Thickener | 0.01 to 30 | 0.05 to 20 | 0.1 to 15 | 0.1 to 10 |
| Misc | total to 100 | total to 100 | total to 100 | total to 100 |
| | Formula 25c | Formula 26c | Formula 27c | Formula 28c |
| Surfactin | 0.5 to 25 | 0.5 to 15 | 1 to 10 | 1 to 7.5 |
| Sophorolipid | 0.5 to 25 | 0.5 to 15 | 1 to 10 | 1 to 7.5 |
| Caring substance | 0.1 to 10 | 0.1 to 7 | 0.5 to 5 | 1 to 5 |
| Thickener | 0.01 to 30 | 0.05 to 20 | 0.1 to 15 | 0.1 to 10 |
| Misc | total to 100 | total to 100 | total to 100 | total to 100 |
| | Formula 29 | Formula 30 | Formula 31 | Formula 32 |
| Surfactin | 0.5 to 25 | 0.5 to 15 | 1 to 10 | 1 to 7.5 |
| Fatty acyl glutamate | 0.5 to 25 | 0.5 to 15 | 1 to 10 | 1 to 7.5 |
| Misc | total to 100 | total to 100 | total to 100 | total to 100 |
| | Formula 29a | Formula 30a | Formula 31a | Formula 32a |
| Surfactin | 0.5 to 25 | 0.5 to 15 | 1 to 10 | 1 to 7.5 |
| Fatty acyl glutamate | 0.5 to 25 | 0.5 to 15 | 1 to 10 | 1 to 7.5 |
| Caring substance | 0.1 to 10 | 0.1 to 7 | 0.5 to 5 | 1 to 5 |
| Misc | total to 100 | total to 100 | total to 100 | total to 100 |
| | Formula 29b | Formula 30b | Formula 31b | Formula 32b |
| Surfactin | 0.5 to 25 | 0.5 to 15 | 1 to 10 | 1 to 7.5 |
| Fatty acyl glutamate | 0.5 to 25 | 0.5 to 15 | 1 to 10 | 1 to 7.5 |
| Thickener | 0.01 to 30 | 0.05 to 20 | 0.1 to 15 | 0.1 to 10 |
| Misc | total to 100 | total to 100 | total to 100 | total to 100 |
| | Formula 29c | Formula 30c | Formula 31c | Formula 32c |
| Surfactin | 0.5 to 25 | 0.5 to 15 | 1 to 10 | 1 to 7.5 |
| Fatty acyl glutamate | 0.5 to 25 | 0.5 to 15 | 1 to 10 | 1 to 7.5 |
| Caring substance | 0.1 to 10 | 0.1 to 7 | 0.5 to 5 | 1 to 5 |
| Thickener | 0.01 to 30 | 0.05 to 20 | 0.1 to 15 | 0.1 to 10 |
| Misc | total to 100 | total to 100 | total to 100 | total to 100 |

Notes:

According to the invention, “Misc” is substantially understood to be water, optionally in combination with another cosmetic carrier, although the cosmetic carrier preferably only comprises water. “Misc” can optionally include a preservative and/or a fragrance. Caring substance: preferably plant oils and/or extracts such as olive oil or aloe vera extract. Thickener: preferably plant thickeners such as xanthan gum, guar gum.

### Examples

The following cleaning agents set out in the tables were produced. The percentages are to be understood as percent by weight, based in each case on the total weight of the cleaning agent.

TABLE 1

| | | Example 2 | Example 3 | | Example 5 |
|---|---|---|---|---|---|
| | Example 1 | Face | Liquid hand | Example 4 | Face |
| INCI or | Mild face | cleanser | soap 3:1 | Mild face | cleanser 3:1 |
| other name | cleanser | concentrate | concentrate | cleanser | concentrate |
| Rhamnolipid | 2 | 6 | 30 | 1 | 3 |
| R-90 | | | | | |
| Surfactin | — | — | — | 1 | 3 |
| Na benzoate | 0.20 | 1 | — | 0.20 | 0.60 |
| Natural | — | 0.20 | 0.60 | 0.10 | 0.30 |
| fragrance | | | | | |
| Water | to 100 | to 100 | to 100 | to 100 | to 100.00 |
| | 100.00 | 100.00 | 100.00 | 100.00 | 100.00 |

TABLE 2

| | | Example 7 | Example 8 | | Example 10 |
|---|---|---|---|---|---|
| | Example 6 | Face | Liquid hand | Example 9 | Face |
| INCI or | Mild face | cleanser | soap 3:1 | Mild face | cleanser 3:1 |
| other name | cleanser | concentrate | concentrate | cleanser | concentrate |
| Rhamnolipid | 1 | 3 | 15 | 1 | 3 |
| R-90 | | | | | |
| Acyl | 1 | 3 | 15 | — | — |
| glutamate | | | | | |
| Sophorolipid | — | — | — | 1 | 3 |
| Soliance S | | | | | |
| Na benzoate | 0.20 | — | — | 0.20 | 0.60 |
| Natural | — | 0.20 | 0.60 | 0.10 | 0.30 |
| fragrance | | | | | |
| Aloe vera | 0.10 | 30 | — | — | — |
| extract | | | | | |
| Water | to 100 | to 100 | to 100 | to 100 | to 100.00 |
| | 100.00 | 100.00 | 100.00 | 100.00 | 100.00 |

TABLE 3

| | | Example 12 | Example 13 | | Example 15 |
|---|---|---|---|---|---|
| | Example 11 | Face | Liquid hand | Example 14 | Face |
| INCI or | Mild face | cleanser | soap 3:1 | Mild face | cleanser 3:1 |
| other name | cleanser | concentrate | concentrate | cleanser | concentrate |
| Rhamnolipid | 1 | 3 | 15 | 1 | 3 |
| R-90 | | | | | |
| Surfactin | 1 | 3 | 15 | 1 | 3 |
| Sophorolipid | — | — | — | 1 | 3 |
| Soliance S | | | | | |
| Na benzoate | 0.20 | — | — | 0.20 | — |
| Natural | — | 0.20 | 0.60 | 0.10 | 0.30 |
| fragrance | | | | | |
| Aloe vera | 0.10 | 30 | — | — | — |
| extract | | | | | |
| Water | to 100 | to 100 | to 100 | to 100 | to 100.00 |
| | 100.00 | 100.00 | 100.00 | 100.00 | 100.00 |

TABLE 4

| | | Example 17 | Example 18 | | Example 20 |
|---|---|---|---|---|---|
| | Example 16 | Face | Liquid hand | Example 19 | Face |
| INCI or | Mild face | cleanser | soap 3:1 | Mild face | cleanser 3:1 |
| other name | cleanser | concentrate | concentrate | cleanser | concentrate |
| Surfactin | 1 | 3 | 20 | 1 | 3 |
| Sophorolipid | 1 | 3 | 20 | 1 | 3 |
| Soliance S | | | | | |
| Na benzoate | 0.20 | — | — | 0.20 | — |
| Natural | — | 0.20 | 0.60 | 0.10 | 0.30 |
| fragrance | | | | | |
| Olive oil | — | — | — | — | 3 |
| KBA | | | | | |
| Water | to 100 | to 100 | to 100 | to 100 | to 100.00 |
| | 100.00 | 100.00 | 100.00 | 100.00 | 100.00 |

TABLE 5

| | | Example 22 | Example 23 | | Example 25 |
|---|---|---|---|---|---|
| | Example 21 | Face | Liquid hand | Example 24 | Face |
| INCI or | Mild face | cleanser | soap 3:1 | Mild face | cleanser 3:1 |
| other name | cleanser | concentrate | concentrate | cleanser | concentrate |
| Surfactin | 1 | 3 | 15 | 1 | 3 |
| Acyl | 1 | 3 | 15 | 1 | 3 |
| glutamate | | | | | |
| Na benzoate | 0.20 | — | — | 0.20 | — |
| Natural | — | 0.20 | 0.60 | 0.10 | 0.30 |
| fragrance | | | | | |
| Guar gum | 0.10 | — | — | — | 50 |
| Water | to 100 | to 100 | to 100 | to 100 | to 100.00 |
| | 100.00 | 100.00 | 100.00 | 100.00 | 100.00 |

TABLE 6

| | | Example 27 | Example 28 | | Example 30 |
|---|---|---|---|---|---|
| | Example 26 | Face | Liquid hand | Example 29 | Face |
| INCI or | Mild face | cleanser | soap 3:1 | Mild face | cleanser 3:1 |
| other name | cleanser | concentrate | concentrate | cleanser | concentrate |
| Rhamnolipid | 1 | 3 | 15 | 1 | 3 |
| R-90 | | | | | |
| Acyl | 1 | 3 | 15 | — | — |
| glycinate | | | | | |
| Sophorolipid | — | — | — | 1 | 3 |
| Soliance S | | | | | |
| Na benzoate | 0.20 | — | — | 0.20 | 0.60 |
| Natural | — | 0.20 | 0.60 | 0.10 | 0.30 |
| fragrance | | | | | |
| Aloe vera | 0.10 | 30 | — | — | — |
| extract | | | | | |

All the compositions in examples 1 to 25 were transferred into an F2 foam pump, applied by means of this foam pump, and subjected to an arm washing test, in which the feel during application, the foaming properties and the washing action were assessed. Each concentrate was diluted to the ratio 3 parts water to 1 part concentrate in the foam dispenser.

An excellent combination of these properties was consistently observed. In particular, the foaming behavior upon application by means of the foam dispenser was extremely satisfactory in all cases. Owing to the antibacterial action of the biosurfactants, it was also possible in particular to provide compositions with no preservatives.

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims and their legal equivalents.

What is claimed is:

1. A cleaning agent system including:
a cleaning agent, 95 wt. % to 100 wt. % of the surfactant content of which is biosurfactant(s), and
a foam dispenser for applying the cleaning agent.

2. The cleaning agent system according to claim 1, wherein the cleaning agent is provided as a concentrate separately from the foam dispenser.

3. The cleaning agent system according to claim 1, wherein 99 wt. % to 100 wt. %, of the surfactant content of the cleaning agent is the biosurfactant(s).

4. The cleaning agent system according to claim 1, wherein the cleaning agent includes a glycolipid, a lipopeptide or a combination thereof as the biosurfactant.

5. The cleaning agent system according to claim 1, wherein the cleaning agent includes a rhamnolipid, a sophorolipid, a mannosylerythritol lipid, a surfactin, a fatty acyl glutamate, a fatty acyl glycinate or a combination thereof as the biosurfactant.

6. The cleaning agent system according to claim 1, wherein the cleaning agent consists of water and the biosurfactant, with the proviso that a preservative, a perfume or fragrance, a caring substance, a thickener or a combination thereof may optionally be included.

7. The cleaning agent system according to claim 1, wherein the cleaning agent is formulated as a cosmetic cleaning agent for the face, body or hands, or as a concentrate thereof.

8. The cleaning agent system according to claim 7, wherein the cleaning agent or cleaning agent concentrate is provided in a bag that can be transferred into the foam dispenser in order to be used.

9. The cleaning agent system according to claim 1, wherein the foam dispenser is a foam pump.
